(19) United States
(12) Patent Application Publication
(10) Pub. No.: US 2010/0069952 A1
(43) Pub. Date: Mar. 18, 2010

(54) CLAMP FOR PROSTATECTOMY

(75) Inventors: Xavier ROUMIER, Saint-Donat (FR); Jérôme MAIRE, Romans (FR)

Correspondence Address: HESLIN ROTHENBERG FARLEY & MESITI PC, 5 COLUMBIA CIRCLE, ALBANY, NY, 12203, US

(73) Assignees: ASPIDE MEDICAL, La Talaudiere (FR); MAIRE JEROME-ROUMIER XAVIER-CENTRE D'UROLOGIE PARISIERE, Bourg De Peage (FR)

(21) Appl. No.: 12/558,993

(22) Filed: Sep. 14, 2009

(30) Foreign Application Priority Data
Sep. 17, 2008 (FR) 0856239

(51) Int. Cl. A61B 17/28

(52) U.S. Cl. 606/207

(57) ABSTRACT

The clamp comprises a body at the rear part extending axially and in a single plane via a fixed long line centring axis to penetrate into the duct of the urethra. The body is provided with a notch for pivoting a prong of longitudinal curvilinear configuration orientated towards said centring axis and in transverse cross-section having the form of a splint over the whole length of the prong. The curvilinear prong being able to grip the outer half-volume of the prostate in order to retain and hold the prostate.

The invention relates to the technical field of appliances and instruments used in surgery and particularly to prostatectomy clamps used when treating cancer of the prostate with a view to the ablation thereof.

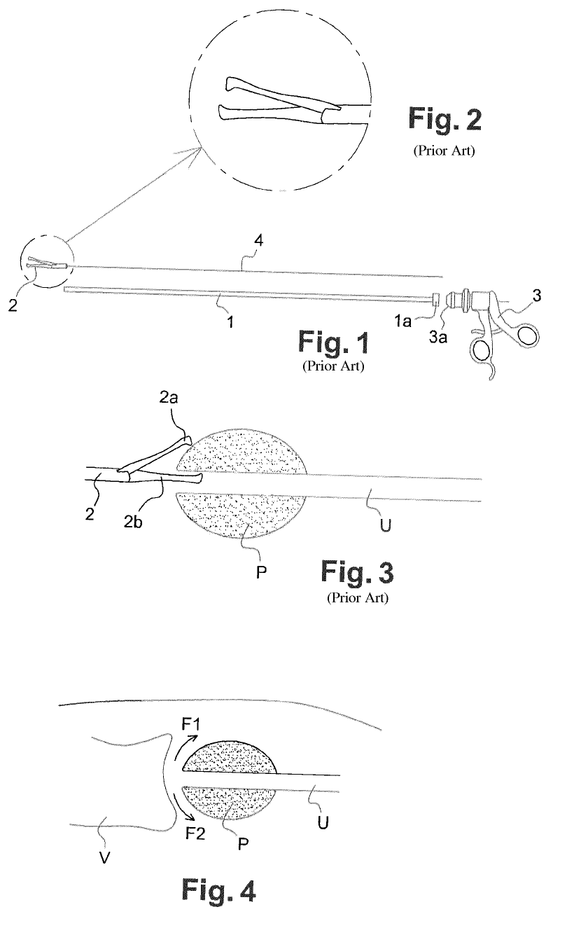

The instruments used to this end are of the same type as those used in laparoscopy and an example is shown in FIG. 1 of the drawings. The instrument involved is thus made out of three parts, a tube (1), a clamp (2) and an operating handle (3). The clamp (2) thus comprises a long line tubular part (4) which is slipped into the tube. The latter has at its rear end a bush (1a) screwed onto the nozzle (3a) of the operating handle. The clamp part (2) comprises at the end two prongs (2a-2b) forming jaws with the opening and closing thereof being controlled by the operating handle. FIG. 2 shows the prior art clamp, as used in laparoscopy, of the type commercially available and known by the name “BABCOCK” from the DUCHATEAU Company. The two prongs (2a-2b) of the clamp are articulated one relative to the other and each have at the end a hook shape which is able to grip itself onto the opposite wall of the prostate. The use of this type of prostatectomy clamp has thus been shown diagrammatically in FIG. 3 in its environment of use.

The prostate has been shown as P, the bladder as V, the duct of the urethra as U. The instrument has been shown diagrammatically in FIG. 4 in the form of a clamp with the two arrows (F1-F2) representing its prongs as they engage and clamp the prostate. A trocar is generally used to pass the instrument tube through.

The problem as posed lies in the fact that the clamps now commercially available and in particular the one known as the “BABCOCK” as shown in FIG. 2, are too small, are of unsuitable geometry and do not have sufficient grip despite their hook-shaped ends. Ablation of the prostate calls for it to be manipulated through the surrounding tissue using rotary and backwards and forwards motions, in order to separate it from the bladder, severing the duct of the urethra. The presence furthermore of a cutting tool around or near to the instrument is an inconvenience and facilitates via the different manipulations poor holding or even a loss of grip of the instrument clamp on the prostate. It is also possible to use this type of clamp as shown in FIG. 3 with one prong entering the duct of the urethra after disengagement thereof, while the other prong hooks itself onto the opposite part of the prostate wall. Given the hook-shaped end configuration of the clamp prongs, this causes damage to the part of the urethra and prostate. Even if the prostate is intended for removal, (the surgeon joins the urethra pipe parts on either side of the prostate using sutures), some damage to the prostate is noted, which is detrimental to the quality of the examinations carried out thereupon for the purposes of analysis and additional research (etc.). Furthermore, the problem is still faced of the external grip of the prostate as mentioned earlier.

Prior art means are thus unsatisfactory in terms of quality, holding, and grip of the prostate with real damage being caused to the prostate itself, which is affected and which is to be removed and analysed.

The applicant has therefore decided to consider the situation and find a solution capable of facilitating the grip and holding of the prostate to facilitate the ablation thereof without however causing any further damage in addition to the after-effects inherent in the tumour.

The solution adopted by the applicant provides a satisfactory response to this problem as posed. Said solution relates exclusively to the clamp part, the other means constituted by the tube and the operating handle for opening and closing the clamp being of the same type as those known.

According to a first inventive feature, the prostatectomy clamp of the type comprising prongs articulated one relative to the other and having an anchored part intended to be secured to the connection tube, itself associated with an operating handle, is remarkable in that it comprises a body at the rear end extending axially and in a single plane via a fixed long line centring axis able to penetrate into the duct of the urethra, and in that the body is provided with a notch for pivoting a prong of longitudinal curvilinear configuration orientated towards said centring axis and in transverse cross-section in the form of a splint over the whole length of the prong, said curvilinear prong being able to surround more than half the outer half-volume of the prostate in order to retain it and hold it.

Said features and yet others will become clear from the remainder of the description.

To establish the object of the invention as shown non-restrictively in the figures in the drawings where:

FIG. 1 is a view of an instrument used in a prostatectomy in accordance with the prior art with a clamp, a connecting tube and an operating handle.

FIG. 2 is a view of a “BABCOCK” prostatectomy clamp according to the prior art.

FIG. 3 is a diagrammatic view showing the position of the clamp relative to the prostate in accordance with the prior art.

FIG. 4 is a view of the clamp according to FIG. 2 inserted into the duct of the urethra and on the opposite wall of the prostate.

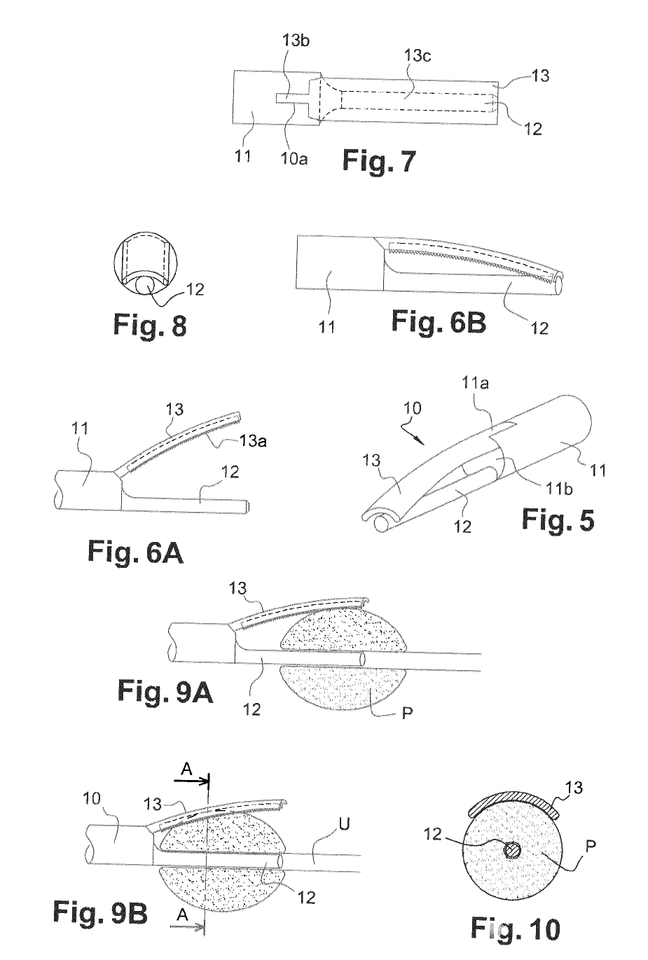

FIG. 5 is a perspective view of the inventive prostatectomy clamp in the closed not-in-use position.

FIGS. 6A-6B are side views of the clamp in accordance with FIG. 5 in the open and closed position.

FIG. 7 is a view from above of the clamp in accordance with FIG. 5.

FIG. 8 is a front view of the clamp in accordance with FIG. 5.

FIGS. 9A, 9B are diagrammatic views showing the position of the inventive clamp with a view to the ablation of the prostate in the position of penetration into the duct of the urethra, and surrounding and retaining the prostate without damage.

FIG. 10 is a cross-section view along the line A-A in FIG. 9.

FIG. 11 shows the clamp with, at the end of the prong thereof, claws.

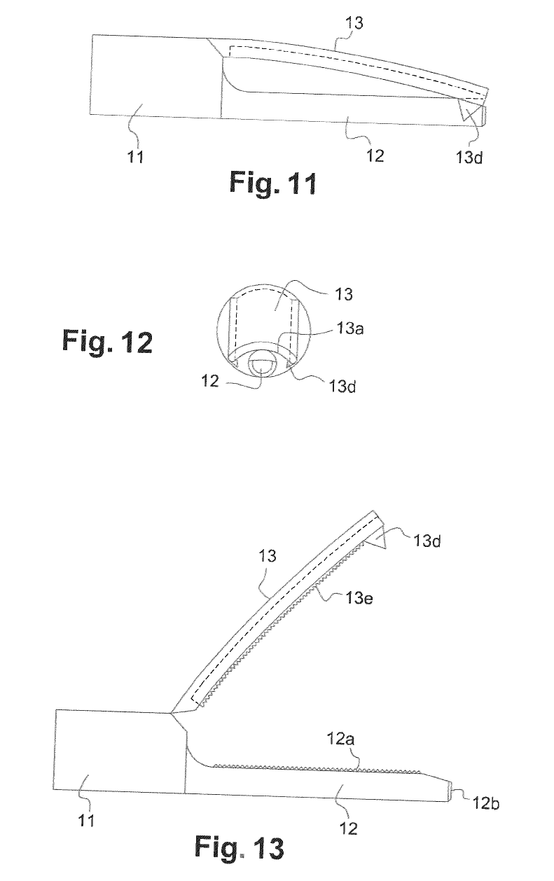

FIG. 12 is a partial front view of the end of the clamp.

FIG. 13 is an alternative view of FIG. 11, the centring axis and the articulated prong having projections.

To make the object of the invention still clearer, it will now be described non-restrictively as illustrated by the figures in the drawings.

The inventive prostatectomy clamp is referred to in its entirety as (10). It has a core body (11) at the rear part which is arranged so as to be secured, in such a way that it can be dismantled, to a tube connected to the operating handle. Said core body (11) is of cylindrical and elongated configuration and has in its front part a tubular centring axis (12) intended to be inserted and positioned in the urethra (U) which is surrounded by the prostate (P). Said long line centring axis (12) being out of alignment over a wide distance in the duct of the urethra (U) relative to the body while being aligned with the latter. Said centring axis is secured and may be added to said body.

In its upper part, the body has a notch (11a) forming a slit leading to the front of said body and allowing the securing and pivoting articulation of a prong (13) of longitudinal curvilinear configuration orientated towards said centring axis and in transverse cross-section in the form of a splint (13a) over the whole length of the prong. Said splint form may be vented by an aperture (13c) (window). Said prong thus has a heel (13b) penetrating into the aforementioned notch, a transverse axis providing connection and articulation. The articulation of said prong is controlled by a conventional return means (not shown) built into said body of the clamp and connected conventionally by the tube to the operating handle in accordance with the practice known in laparoscopy. As can be seen from the drawings, the transverse cross-section of the prong forms a curved splint-shaped profile or is vented by an aperture (13c) (window), with no bumps, or hooked or jagged shapes affording a firm support, and over a great length on the prostate without damaging it. Preferentially, the prong (13) is provided at its front end with two anchoring claws (13d). The centring axis (12) and the prong are fitted with internal facing projections (12a) (13e) to complete the hold on the prostate. The end (12b) of the centring axis may be bevelled.

The length of this articulated prong is such that it can very largely cover the opposite part of the prostate and particularly over a distance such that it covers it beyond its median part, in a continuous assembly.

The articulated prong is in a plane above the centring axis and the two are of substantially the same length away from the front face (11b) of the body.

According to one advantageous feature of the invention, the articulation part of the prong is set back from the front transverse face (11b) of the body so as to create a wide range of movement and allow optimum insertion of the retaining axis into the duct of the urethra. Furthermore, the articulation part itself is protected and is not in contact with the prostate and the duct of the urethra.

The gap between the centring axis (12) and the prong (13) at the point where they are connected to the core body (11) optimizes tightening.

The centring axis (12) may be full or hollow. Its length is adapted to the size of the prostate.

The inventive clamp is mounted onto a support that can be inserted via a conventional surgical trocar.

The advantages are clear from the invention. We should stress first of all the better grip and holding of the prostate due to the axis that penetrates into the duct of the urethra and the particular shape of the prong forming a splint or vented by an aperture (window) which covers a large part of the prostate without damaging it, the assembly allowing and facilitating manipulations and movements in respect of the prostate with a view to the ablation thereof.

Another advantage lies in the better holding and grip of the prostate which is held in particular by a wide longitudinal assembly in the inner part of the prong.

Another advantage lies in the simplicity of the clamp which is made out of a conventional material compatible with its environment of use.

Inventive prostatectomy clamps are adapted according to the size of the prostate of the individuals involved.

1. Prostatectomy clamp comprising prongs articulated one relative to an other and having an anchored part to be secured to a connection tube, the tube being associated with an operating handle, wherein the clamp comprises a body with a rear part extending axially and in a single plane via a fixed long line centring axis to penetrate into a duct of a urethra (U), and the body having a notch for pivoting a prong of longitudinal curvilinear configuration orientated towards said centring axis and in transverse cross-section having a form of a splint over a whole length of the prong, said prong being adapted to grip an outer half-volume of the prostate in order to retain and hold the prostate.

2. Clamp as claimed in claim 1, wherein the long line centring axis is out of alignment over a wide distance in the duct of the urethra relative to the body while being aligned with the body.

3. Clamp as claimed in claim 2, wherein the centring axis is secured and may be added to said body.

4. Clamp as claimed in claim 1, wherein in an upper part, the body has the notch, the notch forming a slit leading to a front of said body and allowing securing and pivoting articulation of the prong and said prong has a heel penetrating into the notch, a transverse axis providing the securing and articulation of the prong.

5. Clamp as claimed in claim 4, wherein the articulation of said prong is controlled by a return means built into said body and connected by the tube to the operating handle.

6. Clamp as claimed in claim 1, wherein the transverse cross-section of the prong forms a curved splint-shaped profile or is vented by an aperture, with no rough patches, or hooked or jagged shapes affording a firm support, and over a great length on the prostate without damaging the prostate.

7. Clamp as claimed in claim 1, wherein the prong is in a plane above the centring axis and offset in order to optimize tightening and the prong and the centring axis are of substantially same length away from a front face of the body.

8. Clamp as claimed in claim 1, wherein an articulation part of the prong is set back from the a transverse front face of the body so as to create a wide range of movement and allow optimum insertion of the centring axis into the duct of the urethra.

9. Clamp, as claimed in claim 1, wherein the prong is provided at its front end with two anchoring claws.

10. Clamp, as claimed in claim 1, wherein the centring axis and the prong are provided with inner facing projections.
